(19) United States
(12) Patent Application Publication
(10) Pub. No.: US 2024/0225719 A1
(43) Pub. Date: Jul. 11, 2024

(54) RADIOFREQUENCY-SHAVER ELECTROSURGICAL INSTRUMENT

(71) Applicant: GYRUS MEDICAL LIMITED, Cardiff (GB)

(72) Inventors: James Alan DICKSON, Cardiff (GB); James AUDSLEY, Cardiff (GB)

(73) Assignee: GYRUS MEDICAL LIMITED, Cardiff (GB)

(21) Appl. No.: 18/403,910

(22) Filed: Jan. 4, 2024

Related U.S. Application Data
(60) Provisional application No. 63/438,110, filed on Jan. 10, 2023.

(51) Int. Cl. A61B 18/14

(52) U.S. Cl. CPC A61B 18/148; A61B 2018/00577; A61B 2218/007

(57) ABSTRACT

A radio-frequency shaver electrosurgical instrument which comprises a thermal barrier located between the central suction lumen and an outer surface of the radiofrequency-shaver electrosurgical instrument, the thermal barrier being disposed around a major portion of the angular extent of the central suction lumen. Without the presence of the thermal barrier, there is a risk of burning the patient if the RF heated saline becomes too hot as the electrosurgical instrument may not be adequately insulated. The thermal barrier prevents or reduces the effect of hot saline heating the outer surface of the instrument, thereby preventing or reducing damage to non-target tissue during use of the radiofrequency function.

### TECHNICAL FIELD

Embodiments of the present disclosure described herein relate to electrosurgical devices, and in particular to a radiofrequency (RF)-shaver combination device.

### BACKGROUND TO THE INVENTION AND PRIOR ART

Electrosurgical instruments provide advantages over traditional surgical instruments in that they can be used for coagulation and tissue sealing purposes. Surgical apparatus used to shave, cut, resect, abrade and/or remove tissue, bone and/or other bodily materials are known. Such surgical apparatus can include a cutting surface, such as a rotating blade disposed on an elongated inner tubular member (or shaft) that is rotated within an elongated outer tubular member (or shaft), each tubular member having a cutting window. The inner and outer tubular members together form a surgical cutting instrument or unit. In general, the elongated outer tubular member includes a distal end defining an opening or cutting window disposed at a side of the distal end of the outer tubular member. The cutting window of the outer tubular member exposes the cutting surface of the inner tubular member (located at a side of the distal end of the inner tubular member) to tissue, bone and/or any other bodily materials to be removed. A powered handpiece is used to rotate the inner tubular member with respect to the outer tubular member while an outer tubular member hub (connected to the proximal end of the outer tubular member) is fixed to the handpiece and an inner tubular member hub (connected to the proximal end of the inner tubular member) is loosely held in place by the powered handpiece.

In some instruments the inner tubular member is hollow and has a cutting window on a side surface of its distal end such that tissue, bone, etc. will be cut or shaved as the cutting window of the inner tubular member aligns with and then becomes misaligned with the cutting window of the outer tubular member as the inner tubular member is rotated within the outer tubular member. In this regard, it can be said that the cutting device removes small pieces of the bone, tissue, etc. as the inner tubular member is rotated within the outer tubular member.

In some instruments a vacuum is applied through the inner tubular member such that the bodily material that is to be cut, shaved, etc. is drawn into the windows of the inner and outer tubular members when those windows become aligned, thereby facilitating the cutting, shaving, etc. of the tissue, which then travels through the inner tubular member due to the suction.

Many times during surgery, the surgeon wishes to apply RF energy to either coagulate bleeding vessels, or ablate tissue in the surgical site without performing cutting with a shaver instrument. This usually is done by withdrawing the surgical instrument and inserting a dedicated RF wand device (for example, an RF ablation wand). However, exchanging the surgical tool for the dedicated RF device is time-consuming. Furthermore, insertion and removal of instruments into the patient can cause trauma and irritation to the passage of the patient, and thus it is desirable to minimize the number of times that surgical instruments need to be withdrawn and inserted/reinserted into the patient.

### SUMMARY OF THE DISCLOSURE

Combining a shaver device with an RF device is not straightforward. An RF Shaver with dual functionality (e.g., RF on one side, shaver on the other) must balance the RF functionality with the shaver functionality at the distal tip of the device. The RF plasma generated by arthroscopic RF ablation probes causes heating of the surrounding saline. This causes hot saline to be created at the active tip of the instrument. In the case of suction-capable RF probes, this heated saline is pulled via a negative pressure source into the distal tip assembly and passes through the shaft and handle of the device via a suction lumen, leaving via outflow tubing at the proximal end.

Due to the conflicting requirements between shaver performance and RF thermal performance, an optimum shaft configuration is difficult to achieve. In brief, an ideal shaver-only device has an inner shaft suction lumen which is as large as possible in order to deal with tissue fragments created by the shearing action in the substantially larger blade cutting window. The inner bore of the inner blade therefore becomes the smallest constriction in the suction system and can therefore be prone to blockage.

An ideal RF-only suction lumen has to carry away ablated tissue fragments, but these fragments are typically much smaller (than fragments created by a shaver-only device) because the hole in the RF tip precludes large chunks of tissue from getting into the suction lumen. Therefore, the active tip or ceramic insulator are the smallest constrictions in the suction system when RF is used with the blade window closed, and the suction lumen in the shaft only has to be marginally larger to carry away tissue debris. The advantage of this smaller suction lumen in an RF device is that it allows there to be space in the overall device construction to create an air gap between the inner suction pathway, which contains heated saline, and the outer shaft which could be in direct patient contact and needs to be cooler. The construction of a typical RF suction probe shaft is such that there is significant thermal insulation present between the heated saline and the outermost surface of the shaft—the surface most likely to be in contact with the skin of the patient during an arthroscopic procedure. This thermal insulation allows the suction of heated saline while ensuring the risk of a burn to the patient remains low. FIG. 1 shows this typical shaft construction which has significant thermal insulation present between the tubular member and the outermost surface of the instrument in the form of an air gap and multiple polymer layers. On the other hand, arthroscopic shaver probes do not typically require this thermal insulation, as the saline being drawn into the device is generally no greater than ambient temperature, and there is therefore no risk of a burn due to heated saline.

As detailed above, a conflict therefore arises when trying to create a ‘best-of-both’ RF shaver, which would preferably have a large suction lumen to maintain shaver performance, and also an air gap for thermal insulation during RF ablation use.

Embodiments of the present invention aim to combine these two very different devices, a shaver device and an RF suction device. Due to the geometric constraints of such a combination, this effectively results in a typical shaver construction with the additional function and components of an RF probe. The RF shaver must therefore manage the risks of heated saline.

The present disclosure addresses the above problem of managing risks of heated saline by providing a thermal barrier within the electrosurgical instrument construction. In this manner, the barrier component is created somewhere within the RF shaver instrument shaft cross section. The barrier component may be in the form of a sealed air gap, or alternatively, may be in the form of a solid material barrier. The barrier component may be located within the static outer blade assembly, the rotating inner blade assembly, or both.

In view of the above, from a first aspect, the present disclosure relates to a radio-frequency shaver electrosurgical instrument comprising: an outer shaft and a concentric relatively rotatable inner shaft, the outer and inner shafts having cutting windows disposed at the distal end thereof, the inner shaft comprising a central suction lumen which, in use, evacuates liquid from the distal end of the electrosurgical instrument; and a radiofrequency active electrode located at the distal end of the electrosurgical instrument (which may be proximal the cutting window). The radiofrequency-shaver electrosurgical instrument is characterised in that the radio-frequency shaver electrosurgical instrument further comprises a thermal barrier located between the central suction lumen and an outer surface of the radiofrequency-shaver electrosurgical instrument (i.e., the outermost surface of the shaft), the thermal barrier being disposed around a major portion of the angular extent of the central suction lumen.

Several advantages are obtained from embodiments according to the above-described aspect. The thermal barrier insulates the outer surface of the shaft of the instrument from the hot saline which passes through the central suction lumen of the instrument during usage of the RF functionality. The hot saline is created at the active tip (distal end of the instrument) and pulled via a negative pressure source into the distal end of the instrument. The thermal barrier prevents the outer surface (which may be in contact with non-target tissue) from reaching dangerously high temperatures.

In some embodiments, the thermal barrier comprises at least one air gap. This is advantageous as air is an excellent insulator. However, it will be appreciated that additional solid material barriers could be used in place of an air gap in other embodiments.

In some embodiments, the thermal barrier comprises one or more of the following: a polymer sleeve, a polymer shaft and a steel shaft.

In some embodiments, the polymer sleeve, polymer shaft and/or steel shaft encloses one or more pockets of air to form the at least one air gap between (a) the polymer sleeve, polymer shaft and/or steel shaft and (b) the inner shaft or the outer shaft. This is advantageous as the polymer sleeve/shaft or steel shaft seals the air gaps. This can be done in different parts of the shaft, e.g., within the rotating inner shaft assembly, the static outer blade assembly, or both. The location of the thermal barrier within the RF-shaver instrument may be chosen according to the desired use of the instrument as different locations have different advantages to consider.

In some embodiments, the thermal barrier is formed between the inner shaft and the outer shaft. This embodiment is advantageous because the RF and shaver performances are unaffected.

In some embodiments, the thermal barrier comprises a polymer sleeve surrounding the inner shaft.

In some embodiments, the thermal barrier is formed between the outer shaft and an outer electrical insulating layer. This embodiment is advantageous because the RF and shaver performances are unaffected, and the design is more space efficient, because it removes the need for a heat shrink layer (ordinarily added as an outer electrical insulating layer) due to the thermal barrier having dual functionality (thermal and electrical insulation).

In some embodiments, the thermal barrier comprises a polymer sleeve which forms the outer electrical insulating layer.

In some embodiments, the thermal barrier comprises stiffening ribs between the outer shaft and the polymer sleeve. This is advantageous as the stiffening ribs ensure the air gaps are maintained, improving the structural integrity of the thermal barrier.

In some embodiments, the thermal barrier is formed between the inner shaft and the central suction lumen. I.e., the thermal barrier is within the inner shaft such that the central suction lumen is limited by the thermal barrier itself. The thermal barrier creates an air gap between the central suction lumen and the inner shaft. This embodiment is advantageous as this air gap is easier to seal during manufacture than other designs.

In some embodiments, the thermal barrier comprises a steel shaft or a polymer shaft.

In some embodiments, the steel or polymer shaft comprises the central suction lumen.

In some embodiments, the steel or polymer shaft is self-supported. This is advantageous as the lack of stiffening ribs means there is no thermal bridging effect such that no hot spots are created.

In some embodiments, the outer shaft has an irregular shaped cross-section arranged to guide a radiofrequency wire delivering energy to the radiofrequency active electrode. This is advantageous as the RF wire is better protected and guided by the non-uniform shaft profile.

In some embodiments, the thermal barrier is disposed around the entirety of the angular extent of the central suction lumen.

In some embodiments, the arrangement is such that, when in use, rotation of the inner shaft within the outer shaft causes a tissue cutting action of the cutting window of the inner shaft interacting with the cutting window of the outer shaft.

In some embodiments, the instrument further comprises an operative shaft having RF electrical connections operably connected to the radiofrequency active electrode.

In some embodiments, the operative shaft further comprises drive componentry operably connected to a rotary shaver arrangement, the rotary shaver arrangement comprising the outer shaft and the inner shaft, to drive the rotary shaver arrangement to operate in use.

From a second aspect, the present disclosure relates to an electrosurgical system, comprising: an RF electrosurgical generator; a suction pump; and an radiofrequency-shaver electrosurgical instrument according to claim 17 or 18, the arrangement being such that in use the RF electrosurgical generator supplies an RF coagulation or ablation signal via the RF electrical connections to the radiofrequency active electrode, and the suction pump supplies suction via the central suction lumen connecting the suction aperture located within the electrode to the suction pump.

From a third aspect, there is provided a method for processing an instrument for surgery, the method comprising: obtaining the radiofrequency-shaver electrosurgical instrument of any of the above aspects; sterilizing the radiofrequency-shaver electrosurgical instrument; and storing the radiofrequency-shaver electrosurgical instrument in a sterile container.

### BRIEF DESCRIPTION OF THE DRAWINGS

Embodiments of the invention will now be further described by way of example only and with reference to the accompanying drawings, wherein:

FIG. 1 is a CAD image showing a typical RF suction probe shaft construction (VAPR Tripolar 90);

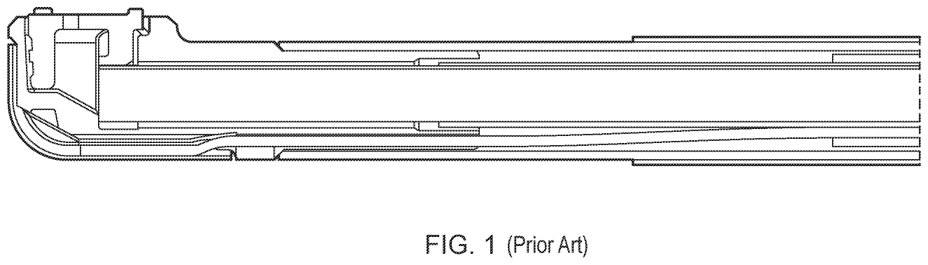

FIG. 2 is a schematic diagram of an electrosurgical system including an electrosurgical instrument;

FIG. 3 is a CAD image showing the shaft construction of an RF shaver design concept;

FIG. 4 is a cross-sectional view of an RF shaver shaft illustrating an embodiment of the present invention;

FIG. 5 is a cross-sectional view of an RF shaver shaft illustrating an embodiment of the present invention;

FIG. 6 is a cross-sectional view of an RF shaver shaft illustrating an embodiment of the present invention;

FIG. 7 is a cross-sectional view of an RF shaver shaft along the line AA of FIG. 9, looking in the direction of the arrows, illustrating an embodiment of the present invention;

FIG. 8 is a cross-sectional view of an RF shaver shaft illustrating an embodiment of the present invention; and

FIG. 9 is a CAD image showing the shaft construction including a thermal barrier in accordance with an embodiment of the present invention.

### DESCRIPTION OF THE EMBODIMENTS

An RF Shaver with opposite sided functionality (RF on one side, shaver on the other) must balance the RF functionality with the shaver functionality at the distal tip of the device. In this configuration, hot saline is created at the active tip and pulled via a negative pressure source (i.e., suction) into the distal tip assembly. In this assembly, the rotating inner tubular member (also referred to as “inner blade”) is positioned in a closed position during RF use such that the cutting windows of the inner tubular member and the static outer tubular member (also referred to as “outer blade”) do not overlap. The inner blade typically has a thin steel construction, which is very effective at cutting tissue, but not an effective thermal insulator.

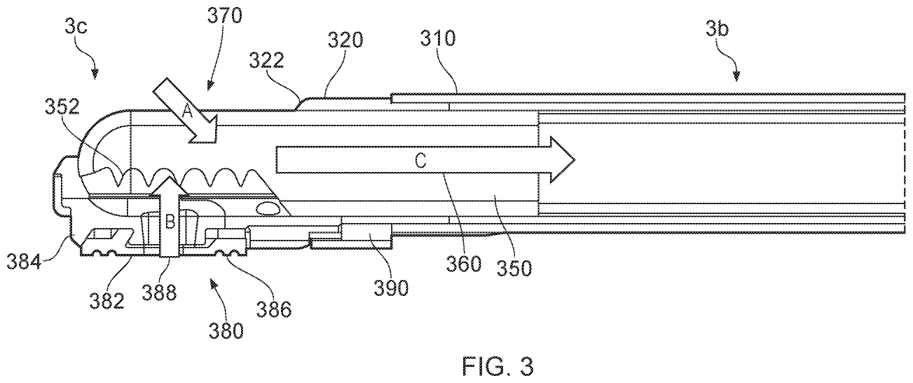

It is more difficult to implement thermal insulation into the RF shaver shaft design than in previous RF suction probes. This is because the outer shaft inner diameter is taken up almost entirely by the inner shaft, which is the main suction path for the heated saline. This results in a device construction shown in FIG. 3 that does not include the air gap and polymer insulation layers present on typical RF suction probes. This reduction in thermal insulation may lead to a greater risk of high outer shaft temperatures, which in turn may lead to risk of patient burn at the point of skin-contact.

Embodiments of the present invention provide a shaft for an RF-shaver device which has a thermal barrier component which reduces the effect of the hot saline passing through the central suction lumen (which runs through the inner blade) on heating the inner blade.

The thermal barrier component is advantageous for RF-shaver electrosurgical instruments which combine rotary shaver arrangements and RF electrode arrangements, where suction is used to remove RF heated saline from the surgical site. Without the presence of the thermal barrier component, there is a risk of burning the patient if the RF heated saline becomes too hot as the electrosurgical instrument may not be adequately insulated. The thermal barrier component prevents or reduces the effect of hot saline heating the inner tubular member, thereby preventing or reducing damage to non-target tissue during use of the radiofrequency function.

In more detail, a dual function RF-shaver electrosurgical instrument has a rotary arrangement of two concentric cylindrical shafts, an inner shaft (also referred to as an inner tubular member) and an outer shaft (also referred to as an outer tubular member).

The inner shaft rotates relative to the outer shaft. Both the outer and inner shafts have a cutting window at their distal ends. Suction is applied along a suction path which extends from the proximal end of the instrument through the lumen of the inner shaft and through the cutting window. The suction draws tissue to be cut into the cutting window, where it is severed by the rotating inner shaft. The inner shaft may have serrated edges along its cutting window. The tissue is then suctioned into the lumen and taken away from the surgical site. In addition to the shaver capability, the instrument also has RF capabilities by virtue of an RF electrode mounted on the opposite side of the operative shaft to the outer shaft's distal cutting window. The RF electrode may be used to cut, coagulate, desiccate or fulgurate tissue. Within the electrode is a suction aperture which is also connected to the lumen of the inner shaft. A suction path extends from the lumen through the suction aperture in the electrode. This suction path is an alternative to the suction path which extends through the cutting window of the shaver side. When the surgeon wishes to apply suction via the suction aperture on the RF side, the cutting window of the shaver side is closed by rotating the inner shaft such that the cutting windows of the inner and outer shaft are misaligned. The RF energy applied by the electrode may result in RF heated plasma which in turn heats saline and/or tissue. This heated material (saline and/or ablated tissue) is then suctioned away from the surgical site through the suction aperture within the electrode. The heated material therefore travels down the suction path through the lumen of the inner shaft. Without the presence of the thermal insulating component of embodiments of the present invention, this hot material may heat the outermost surface of the shaft of the instrument to high temperatures. Such high temperatures could potentially burn the patient.

### The Electrosurgical System

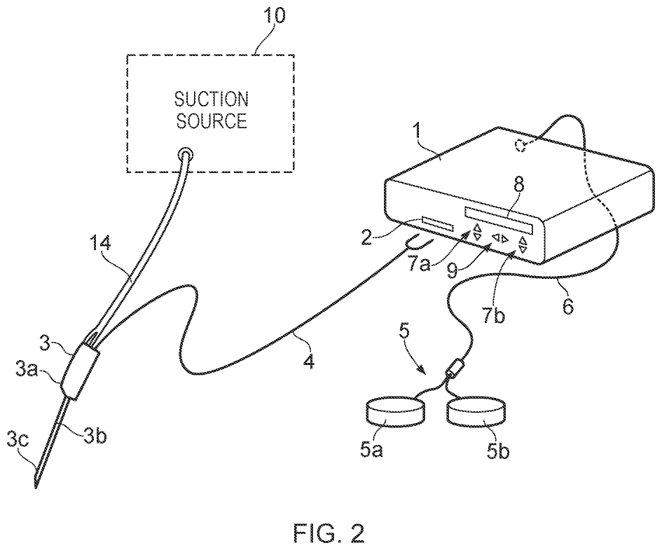

Referring to the drawings, FIG. 2 shows electrosurgical system including an electrosurgical generator 1 having an output socket 2 providing an RF output, via a connection cord 4, for an electrosurgical instrument 3. The instrument 3 has suction tubes 14 which are connected to a suction pump 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

### The Electrosurgical Instrument

The RF-shaver instrument 3 includes a proximal handle portion 3a, a hollow shaft 3b extending in a distal direction away from the proximal handle portion, and a distal end effector assembly 3c at the distal end of the shaft. A power connection cord 4 connects the instrument to the RF generator 1. The instrument may further be provided with activation buttons (not shown), to allow the surgeon operator to activate either the mechanical cutting function of the end effector, or the electrosurgical functions of the end effector, which typically comprise coagulation or ablation.

FIG. 3 shows an example of the distal end effector assembly 3c in more detail. The distal end effector 3c has two sides to it, the shaver side 370 and the RF side 380.

The inner shaft 350 is co-axially disposed within an outer shaft 320. The outer shaft 320 acts as the RF return. The outer shaft has an outer electrical insulation layer 310 (which may be a heat shrink layer) around it. The outer shaft 320 has a larger diameter than the inner shaft 350. The inner shaft 350 is a tubular member having a proximal end and a distal end, with cutting window 352 disposed at a side of its distal end. The outer shaft 320 is also a tubular member having a proximal end and a distal end, with cutting window 322 disposed at a side of its distal end. The outer shaft 320 is static (i.e., it does not rotate). The inner shaft 350 is rotatably disposed inside of the outer shaft 320 such that the surgical instrument 3 cuts tissue by rotating the inner shaft 350 within the outer shaft 320 while a vacuum is applied through the hollow lumen of the inner shaft 350 (the suction lumen 360) to draw the tissue into the cutting windows 352 and 322 and sever the tissue by rotation of the inner shaft.

The RF side 380 of the electrosurgical instrument 3 comprises an electrode assembly comprising an active electrode for tissue treatment (“active tip”) 382 received in a ceramic insulator 384. The RF active tip 382 is connected to RF wire 390 which is in turn connected to connection cord 4, which connects to the generator 1. The active tip 382 is provided with projections 386 to concentrate the electric field at those locations. The projections 386 also serve to create a small separation between the planar surface of the active electrode 382 and the tissue to be treated. This allows conductive fluid to circulate over the planar surface and avoids overheating of the electrode or the tissue. The active tip 382 of the instrument is provided with a suction aperture 388, which is the opening to a lumen within an inner shaft 350.

In more detail, when the RF side 380 is to be used as a suction tool by applying a vacuum through the lumen within the inner shaft 350, the inner shaft 350 (which acts as a cutting blade) is stopped from rotating and the cutting windows 352 and 322 are misaligned with each other, i.e. closing the cutting windows, (as is the case in FIG. 3) so that the vacuum is applied through the suction path connecting the suction aperture 388 to the suction pump 10 via the lumen (i.e. the suction path defined by arrows B and C) to transport fluids to and from the active tip 382.

In contrast, when the shaver side 370 is in use for a cutting operation, suction flows via the suction path defined by arrows A and C, i.e. through the cutting windows to the lumen.

The inner and outer shafts 350 and 320 are made from a sterilisable material. For example, the sterilisable material may be a metal such as steel, e.g., stainless steel.

### Thermal Barrier Component

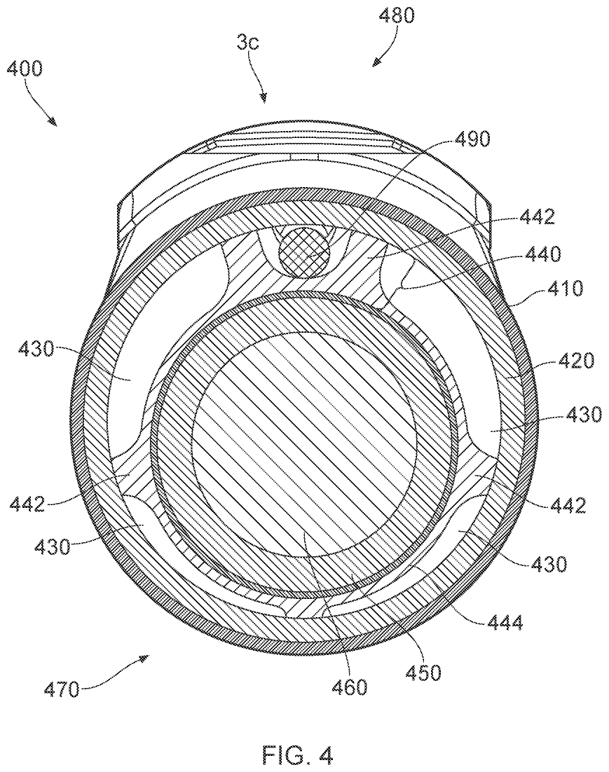

Embodiments of the present invention provide a shaft for an RF-shaver device which has a thermal barrier component. Embodiments of the present invention will be described in more detail with reference to FIGS. 4 to 8.

FIGS. 4-8 show the distal end of an RF-shaver instrument. The distal end effector assembly 3c is the same as the end effector assembly 3c described in relation to FIG. 3. However, the shaft assembly of FIGS. 4-8 differs from that shown in FIG. 3 (see reference numeral 3b). The shaft assemblies of FIGS. 4-8 mainly differ from that of FIG. 3 in that FIGS. 4-8 additionally show a thermal barrier 440, 540, 640, 740, 840 which is the subject of the present disclosure. The thermal barrier component 440, 540, 640, 740, 840 is located within the shaft assembly of the RF shaver instrument. The thermal barrier may be within the static outer blade assembly, the rotating inner blade assembly, or both. The thermal barrier may comprise a steel shaft, a polymer shaft/sleeve, stiffening ribs, and/or one or more air pockets.

### First Embodiment

Referring to FIG. 4, the shaft 400 is similar to the shaft 3b shown in FIG. 3 and like reference numerals are used accordingly (e.g., the end effector has two sides to it, the shaver side 470 and the RF side 480, compared with shaver side 370 and RF side 380 of FIG. 3). The differences between the shaft 3b shown in FIG. 3 and the shaft 400 shown in FIG. 4 are explained below.

FIG. 4 shows an example of a first embodiment of the invention which, unlike shaft 3b of FIG. 3, has a thermal barrier 440 within the RF-shaver construction between the static outer blade 420 and the rotating inner blade 450. The thermal barrier 440 reduces heat transferred thermally to the outer surface of the shaft, via insulating air pockets 430 sealed between the outer 420 and inner 450 shaft.

When the RF side 480 is in use, the hollow suction lumen 460 is filled with hot saline being suctioned away from the treatment site. The thermal barrier 440 comprises multiple air gaps 430 between the inner 450 and outer 420 shafts which act to insulate the outer surface of the shaft from the heat of the saline in the suction lumen 460. The thermal barrier 440 comprises two main structural components—stiffening ribs 442 and a polymer sleeve 444 (may also be referred to as a polymer shaft) surrounding the inner shaft 450. The structure of the thermal barrier 440 acts as a spacer and maintains a gap between the inner 450 and outer 420 shafts. The gap maintained between the inner 450 and outer 420 shafts is supported by the plurality of stiffening ribs 442. The structural components of the thermal barrier 440 are preferably made from polymer material.

To summarise, the advantages of the first embodiment are that it creates a thermal barrier 440 using air pockets/gaps 430. Both the RF performance and shaver performance of the electrosurgical instrument are unaffected.

### Second Embodiment

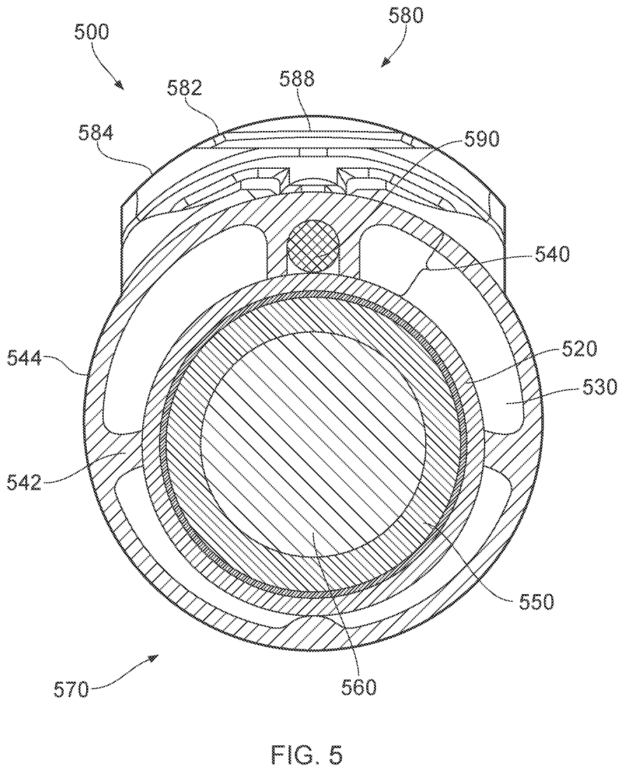

FIG. 5 shows an example of a second embodiment of the invention which has a thermal barrier 540 within the RF-shaver construction. Shaft 500 of FIG. 5 is similar to shaft 400 of FIG. 4 and like reference numbers are used accordingly. The difference between the first embodiment and the second embodiment is that in the first embodiment, the thermal barrier 440 is located between the inner 450 and the outer 420 shafts, whereas in the second embodiment, the thermal barrier 540 is located outside the outer shaft 520 and the polymer sleeve component 544 of the thermal barrier 540 also forms the outer electrical insulation layer. Thus, the thermal barrier 540 is formed between the outer shaft 520 and the outer electrical insulation layer. Air pockets 530 are sealed outside the outer shaft 520 by the polymer sleeve 544.

The thermal barrier 540 comprises multiple air gaps 530 between the polymer sleeve 544 and the outer shaft 520. A plurality of stiffening ribs 542 maintain a gap between the outer shaft 520 and the polymer sleeve 544 (which forms the outer electrical insulation layer), creating a secondary lumen between the outer shaft 520 and the polymer sleeve 544. The structural components of the thermal barrier 540 are preferably made from polymer material.

Compared to the first embodiment, the second embodiment has the additional advantage that it is space efficient because it removes the need for a heat shrink layer due to the polymer component having dual functionality of thermal and electrical insulation.

### Third Embodiment

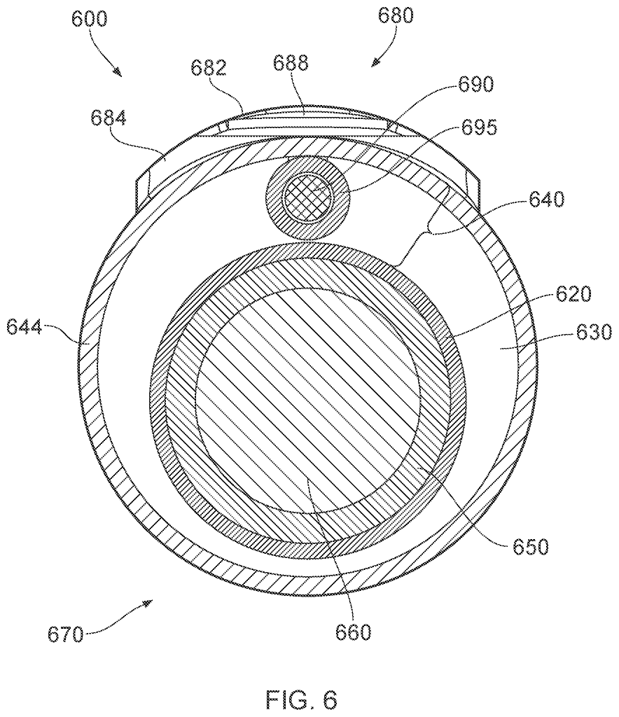

FIG. 6 shows an example of a third embodiment of the invention which has a thermal barrier 640 within the RF-shaver construction. Shaft 600 of FIG. 6 is similar to shafts 400 and 500 of FIGS. 4 and 5 and like reference numbers are used accordingly. Similarly to the second embodiment, the polymer shaft component (also referred to as a polymer sleeve) of the thermal barrier 640 forms the outer electrical insulation layer and is located outside the outer shaft 620. Unlike the earlier embodiments described above, the third embodiment illustrated by FIG. 6 does not have stiffening ribs to support the polymer shaft part of the thermal barrier 640. Thus, the thermal barrier 640 of the third embodiment is made up of one main structural component—the polymer shaft. This is advantageous because the lack of stiffening ribs prevents a thermal bridging effect. The thermal bridging effect arises when stiffening ribs act as thermal bridges and create localised hot spots on the outer shaft. The lack of thermal bridges also means the example illustrated in FIG. 6 only has one air pocket 630. In the third embodiment, the RF wire 690 is preferably constrained by a tube 695 to guide the wire from the hub at the proximal handle portion 3a to the end effector because there is no structural support for the RF wire 690 from the outer shaft 620.

Compared to the second embodiment, the third embodiment has the additional advantage that there is no thermal bridging effect as the polymer sleeve/shaft is unsupported (in contrast to the earlier embodiments which have stiffening ribs which create localised hot spots on the outer surface of the shaft). However, the lack of stiffening ribs has the disadvantage that the polymer shaft is liable to collapse the air gap if it is loaded. Further, as the outer shaft 620 does not support the RF wire 690, the additional component of the tube 695 to guide the RF wire 690 is preferable.

### Fourth Embodiment

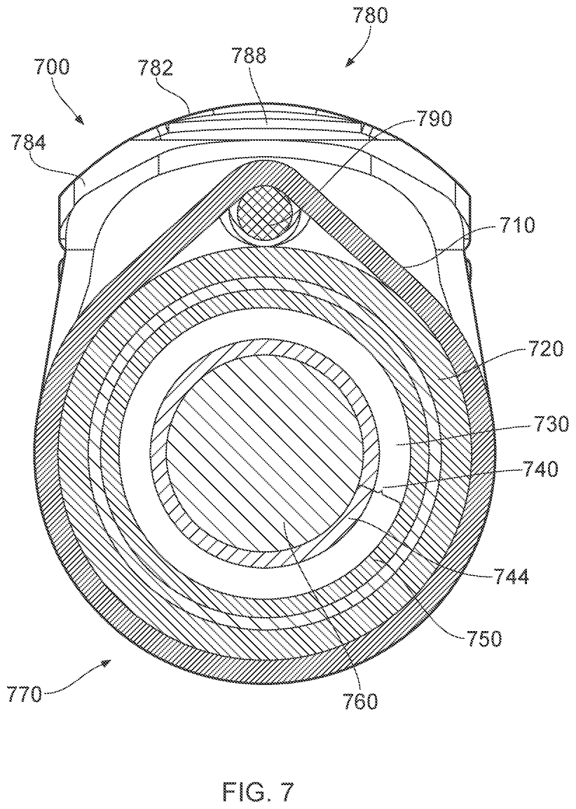

FIG. 7 shows an example of a fourth embodiment of the invention which has a thermal barrier 740 within the RF-shaver construction. Shaft 700 of FIG. 7 is similar to shafts 400, 500 and 600 of FIGS. 4-6 and like reference numbers are used accordingly. The fourth embodiment illustrated by FIG. 7 differs from the earlier embodiments in that in the fourth embodiment, in addition to the inner and outer shafts there is an additional “insulation” shaft 744 which creates the air gap 730. The thermal barrier 740 comprises the insulation shaft 744 which is analogous to the polymer shaft of the previously described embodiments.

In FIG. 7, the insulation shaft 744 is co-axially disposed within the inner shaft 750 and the air gap 730 is created between the insulation shaft 744 and the inner shaft 750. The insulation shaft 744 has a hollow lumen which acts as a suction lumen 760. When the RF side 770 is in use, the lumen 760 is filled with hot saline being suctioned away from the treatment site.

Additionally or alternatively, the insulation shaft 744 (or a further insulation shaft) could be co-axially disposed between the inner shaft 750 and the outer shaft 720 such that the air gap 730 is created between the inner shaft 750 and the insulation shaft 744. In this case, the inner shaft 750 has a lumen which acts as a suction lumen 760. When the RF side 770 is in use, the lumen 760 is filled with hot saline being suctioned away from the treatment site.

Insulation shaft 744 is self-supporting and may be a polymer shaft or a steel shaft. FIG. 7 shows an example where there is only one air gap/pocket 730. The air gap 730 and the insulation shaft 744 form the thermal barrier 740. Because the insulation shaft 744 is self-supporting, the fourth embodiment does not need stiffening ribs to support the insulation shaft 744. Thus, the thermal barrier 740 of the fourth embodiment is made up of one main structural component—the insulation shaft 744. This is advantageous because the lack of stiffening ribs prevents the thermal bridging effect described above. The insulation shaft may be formed from a polymer material or steel. The air gap 730 acts as a secondary lumen between the inner shaft 720 and the insulation shaft 744.

Compared to the third embodiment, the fourth embodiment has the additional advantage that the thermal barrier is created using concentric air pockets—the air gap 730 is uniform which keeps the temperature of the outer surface of the shaft uniform. The air gap 730 of the fourth embodiment is also easier to seal during manufacture than the earlier embodiments because there is no need to seal the air gap around the active wire 790. There is no thermal bridging effect as the insulation shaft 744 is self-supporting (in contrast to the first and second embodiments which have stiffening ribs which create localised hot spots on the outer surface of the shaft). Further, the self-supporting shaft 744 is also not liable to collapse the air gap if it is loaded, unlike the third embodiment.

### Fifth Embodiment

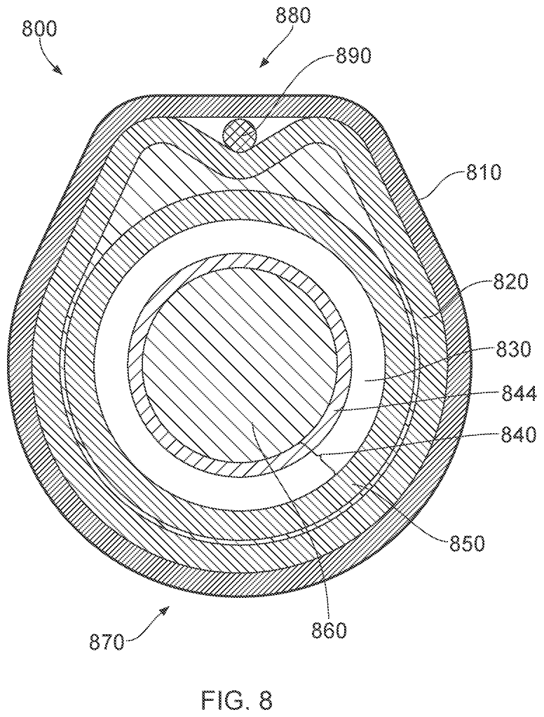

FIG. 8 shows an example of a fifth embodiment of the invention which has a thermal barrier 840 within the RF-shaver construction. FIG. 8 is similar to FIGS. 4-7 and like reference numbers are used accordingly. The fifth embodiment is identical to the fourth embodiment, except for the outer shaft 820 and the outer electrical insulation layer 810. In the fourth embodiment, the outer shaft 720 is a regular uniform cylindrical shape with a circular cross-section and the outer electrical insulation layer 710 encapsulates the outer shaft and the RF wire 790, thereby having an irregular shape with a teardrop-shaped cross-section. In the fifth embodiment, the outer shaft 820 is non-uniformly shaped. Instead of being a regular cylindrical shape with a circular cross-section, the outer shaft 820 of the fifth embodiment is irregularly shaped to support the underneath of the RF wire 890. This has the effect of the outer electrical insulation layer 810 being flat across the RF side 880 (opposed to pointed as in the fourth embodiment). The fifth embodiment is therefore advantageous over the fourth embodiment as the RF wire is better protected and guided by the non-uniform shaft profile of the outer shaft 820.

The fourth and fifth embodiments are inner blade insulation concepts. In these embodiments a cylinder of air is sealed between two tubes, this may be achieved using laser welding or adhesives.

### Summary of the Thermal Barrier Component

The thermal barrier 440, 540, 640, 740, 840 may comprise an air gap 430, 530, 630, 730, 830 such that the thermal barrier 440, 540, 640, 740, 840 encloses one or more pockets of air (the air gap(s)). This is advantageous because air is a better thermal insulator than most solid materials, and air is also lighter than a solid material. However, any of the above embodiments may be modified to fill the air gap 430, 530, 630, 730, 830 with solid material. Such a solid material should be a thermally insulating material, for example, a ceramic such as Alumina or Zirconia, or any blend could be used. Alternatively or additionally, various heat resistant polymers could be used.

A key aspect of the invention is that the thermal barrier component is located within the shaft of the RF-shaver (either within the static outer blade assembly, the rotating inner blade assembly, or both) to prevent or reduce heat from the hot saline in the suction lumen being transferred to the outer surface of the shaft which may be in direct contact with non-target tissue during treatment.

The presence of the thermal barrier component results in a substantial reduction in the outer surface temperature of the RF-shaver electrosurgical instrument.

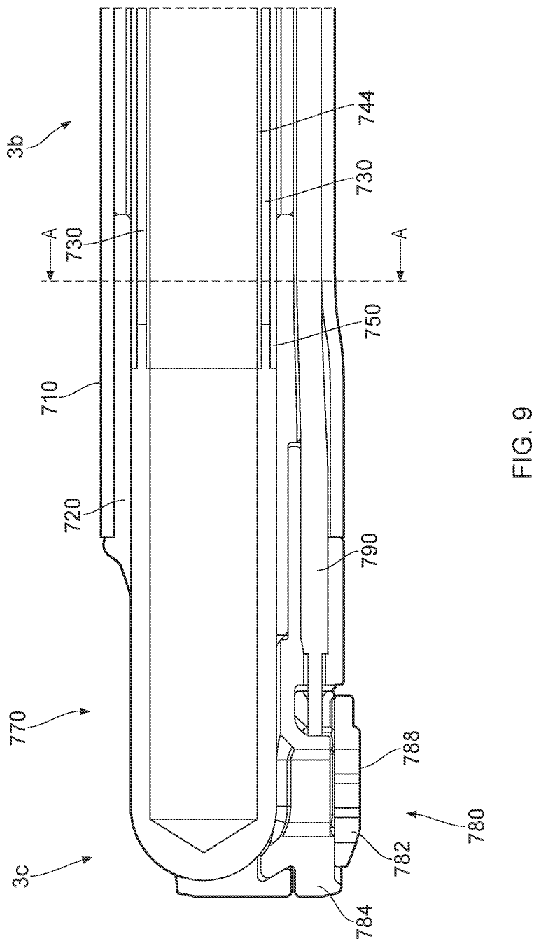

FIG. 9 shows a side view of an RF-shaver instrument with the thermal barrier in place. For this example, the thermal barrier is the thermal barrier of the fourth embodiment 700 shown in FIG. 7 and described above. FIG. 7 is a cross-sectional view along the line AA of FIG. 9, looking in the direction of the arrows. Like reference numerals have been used accordingly. The thermal barrier is located in the shaft 3b of the instrument and is comprised of the insulation shaft 744 which creates the air gap 730 between the insulation shaft 744 and the inner shaft 750, as described in detail above.

### Manufacturing

If the thermal barrier component is made of polymer, an adhesive at either end may be used to affix and seal it in place. If steel tubes are used to create the thermal barrier, welding may be used to provide a sufficient seal and mechanical integrity.

An additional option to locate and seal the thermal barrier for the proximal end of the shaft construction may use the plastic hub component. By over-moulding the plastic hub component onto either the inner or outer shaft, this process could both locate and seal the thermal barrier at the proximal end.

Another option to create a seal and locate barrier components (either polymer or steel) may use o-rings at each end of the air gap.

The thermal barrier component itself may be drawn or machined if steel. If a complex shape is required, hydroforming may be employed.

If the thermal barrier is formed from polymer, extrusion or injection moulding may be used.

### Reprocessing

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

Various modifications whether by way of addition, deletion, or substitution of features may be made to above-described embodiment to provide further embodiments, any and all of which are intended to be encompassed by the appended claims.

1. A radio-frequency shaver electrosurgical instrument comprising:
an outer shaft and a relatively rotatable inner shaft, the outer and inner shafts having cutting windows disposed at the distal end thereof, the inner shaft comprising a central suction lumen which, in use, evacuates liquid from the distal end of the electrosurgical instrument; and
a radiofrequency active electrode located at the distal end of the electrosurgical instrument;
wherein the radio-frequency shaver electrosurgical instrument further comprises a thermal barrier located between the central suction lumen and an outer surface of the radiofrequency-shaver electrosurgical instrument, the thermal barrier being disposed around a major portion of the angular extent of the central suction lumen.

2. The radiofrequency-shaver electrosurgical instrument of claim 1, wherein the thermal barrier comprises at least one air gap.

3. The radiofrequency-shaver electrosurgical instrument of claim 1, wherein the thermal barrier comprises one or more of the following: a polymer sleeve, a polymer shaft and a steel shaft.

4. The radiofrequency-shaver electrosurgical instrument of claim 3, wherein the polymer sleeve, polymer shaft and/or steel shaft encloses one or more pockets of air to form the at least one air gap between (a) the polymer sleeve, polymer shaft and/or steel shaft and (b) the inner shaft or the outer shaft.

5. The radiofrequency-shaver electrosurgical instrument of claim 1, wherein the thermal barrier is formed between the inner shaft and the outer shaft.

6. The radiofrequency-shaver electrosurgical instrument of claim 5, wherein the thermal barrier comprises a polymer sleeve surrounding the inner shaft.

7. The radiofrequency-shaver electrosurgical instrument of claim 1, wherein the thermal barrier is formed between the outer shaft and an outer electrical insulating layer.

8. The radiofrequency-shaver electrosurgical instrument of claim 7, wherein the thermal barrier comprises a polymer sleeve which forms the outer electrical insulating layer.

9. The radiofrequency-shaver electrosurgical instrument of claim 6, wherein the thermal barrier comprises stiffening ribs between the outer shaft and the polymer sleeve.

10. The radiofrequency-shaver electrosurgical instrument of claim 1, wherein the thermal barrier is formed between the inner shaft and the central suction lumen.

11. The radiofrequency-shaver electrosurgical instrument of claim 5, wherein the thermal barrier comprises a steel shaft or a polymer shaft.

12. The radiofrequency-shaver electrosurgical instrument of clam 11, wherein the steel or polymer shaft comprises the central suction lumen.

13. The radiofrequency-shaver electrosurgical instrument of claim 11, wherein the steel or polymer shaft is self-supported.

14. The radiofrequency-shaver electrosurgical instrument of claim 10, wherein the outer shaft has an irregular shaped cross-section arranged to guide a radiofrequency wire delivering energy to the radiofrequency active electrode.

15. The radiofrequency-shaver electrosurgical instrument of claim 1, wherein the thermal barrier is disposed around the entirety of the angular extent of the central suction lumen.

16. The radiofrequency-shaver electrosurgical instrument of claim 1, wherein the arrangement is such that, when in use, rotation of the inner shaft within the outer shaft causes a tissue cutting action of the cutting window of the inner shaft interacting with the cutting window of the outer shaft.

17. The radiofrequency-shaver electrosurgical instrument of claim 1, further comprising an operative shaft having RF electrical connections operably connected to the radiofrequency active electrode.

18. The radiofrequency-shaver electrosurgical instrument of claim 17, wherein the operative shaft further comprises drive componentry operably connected to a rotary shaver arrangement, the rotary shaver arrangement comprising the outer shaft and the inner shaft, to drive the rotary shaver arrangement to operate in use.

19. The radiofrequency-shaver electrosurgical instrument of claim 8, wherein the thermal barrier comprises stiffening ribs between the outer shaft and the polymer sleeve.

20. An electrosurgical system, comprising:
an RF electrosurgical generator;
a suction pump; and
a radio-frequency shaver electrosurgical instrument comprising:
an outer shaft and a relatively rotatable inner shaft, the outer and inner shafts having cutting windows disposed at the distal end thereof, the inner shaft comprising a central suction lumen which, in use, evacuates liquid from the distal end of the electrosurgical instrument; and a radiofrequency active electrode located at the distal end of the electrosurgical instrument;
wherein the radio-frequency shaver electrosurgical instrument further comprises a thermal barrier located between the central suction lumen and an outer surface of the radiofrequency-shaver electrosurgical instrument, the thermal barrier being disposed around a major portion of the angular extent of the central suction lumen, and an operative shaft having RF electrical connections operably connected to the radiofrequency active electrode
the arrangement of the electrosurgical system being such that in use the RF electrosurgical generator supplies an RF coagulation or ablation signal via the RF electrical connections to the radiofrequency active electrode, and the suction pump supplies suction via the central suction lumen connecting the suction aperture located within the electrode to the suction pump.
